(19) United States
(12) Patent Application Publication
(10) Pub. No.: US 2005/0226788 A1
(43) Pub. Date: Oct. 13, 2005

(54) Air scenting apparatus

(76) Inventors: Michael Hrybyk, Warrington (GB); Deborah Clayton, Warrington (GB)

Correspondence Address: THE WEBB LAW FIRM, P.C., 700 KOPPERS BUILDING, 436 SEVENTH AVENUE, PITTSBURGH, PA, 15219, US

(21) Appl. No.: 10/508,591

(22) PCT Filed: Mar. 21, 2003
(86) PCT No.: PCT/GB03/01227

§ 371 Date: May 10, 2005

(30) Foreign Application Priority Data
Mar. 22, 2002 (GB) 0206806.2

(51) Int. Cl. A61L 9/012

(52) U.S. Cl. 422/124

(57) ABSTRACT

An air scenting apparatus comprising; a chamber, the chamber having at least one air inlet aperture and at least one air outlet aperture; a fan arranged to draw air into the chamber through an inlet aperture and out via an air outlet aperture; and a vessel containing a scenting medium arranged to lie at least partially in the air stream between the air inlet and air outlet apertures. The fan may be a centrifugal fan. The apparatus may be remotely controlled and may enable more than one scent to be delivered.

The present invention relates to air scenting apparatus.

Air scenting has become, in recent years, an important aspect of marketing and healthcare therapies. The presence of a scent in a room has been shown to have positive effects on the occupants of a room in terms of their psychological state. In particular, air scenting has been used in hospitals to promote feelings of well being to patients recovering from a variety of conditions. Moreover, scents have been employed as a ‘subliminal’ tool in selling products. A particularly good example of this is the smell of fresh baked bread or ground coffee when trying to sell a house, or the smell of fresh flowers or food produce in supermarkets. Consequently, the use of air scenting has become a commonplace tool in many areas of life.

Normally to produce a scent in a large volume of space, in for example, a home, shop or hospital ward, air scenting apparatus are used. Generally speaking, air scenting apparatus fall into three categories namely; burnt incense, oil burners and aerosols. The object of all three types however, is to produce a fine dispersion of scented particles in the air.

In the case of incense, the substance is often in the form of a coating on a combustible stick. The stick is lit using a flame and the substance slowly smoulders, releasing the scent as smoke particles. Often however, the scent is overpowered by a smoky smell and may sometimes be acrid or irritating to the eyes or nose. Moreover, the smoke may leave unsightly or pollutant residues on clothing, furniture, carpets or the building itself.

The second solution, the oil burner, comprises a volatile scent held in close proximity to a heat source. The heat causes the scent, usually in the form of an oil or solution to evaporate, and the vapour particles are allowed to disperse throughout the room. Again, similar problems to the incense may be encountered.

A preferred solution, in recent years has been to form an aerosol, that is to say fine liquid droplets in air, from a liquid or gelatinous scenting medium. This can be achieved by forcing the liquid through a fine annulus or aperture, thereby breaking a stream of the liquid into a spray of finely dispersed droplets. Improvements in nozzle design have led to the production of pseudo-stable aerosols. Alternatively, the venturi principle may be employed whereby a fast current of air is passed over the liquid scenting medium, causing a localised pressure drop over the liquid. The air current may be produced by a rotor or fan, or is sometimes provided by an on-board compressed air supply. The pressure drop may either cause the scenting medium to evaporate and disperse as a vapour spontaneously, or may be used to draw a stream of liquid through a fine nozzle to generate a spray.

The main problems encountered with the nozzle-type aerosol systems is that the dispersion of the aerosol is difficult to control and may lead to over use or under use of the scenting medium. Clogging or contamination of the nozzles is commonplace and makes it difficult to change scents quickly easily. Moreover, when changing scents, residual scent or mixing may occur for a period after having changed to the new scent.

The present invention operates on a combination of the ‘pressure drop to evaporate’ and the venturi principles.

It is an object of the present invention to provide an air scenting apparatus based on the ‘pressure drop to evaporate’ principle, which optimises scenting medium usage, enables rapid and efficient dispersion of scent into the air and provides easier means for changing the scent.

Accordingly, the present invention provides an air scenting apparatus comprising;
a chamber, the chamber having at least one air inlet aperture and at least one air outlet aperture; a fan arranged to draw air into the chamber through an inlet aperture and out via an air outlet aperture; and a vessel containing a scenting medium arranged to lie at least partially in the air stream between the air inlet and air outlet apertures.

The apparatus is preferably contained in a body which may have any one or more of the following features; a carrying handle, a mounting bracket or brackets, legs, adjusters for varying the orientation of the apparatus and/or tamper-proof guards.

The body contains a chamber, which is sized and shaped to completely accommodate the fan. Preferably, there is a minimal clearance around the fan in its non-working axis, that is to say minimum radial clearance for a screw-type fan and minimum axial clearance for a centrifugal-type fan. There is also present in the chamber, at least one of each of an inlet and an outlet aperture, although preferably, and for simplicity of design and manufacture there is just one of each.

The inlet and outlet apertures may have guards to prevent ingress of unwanted objects or body-parts. Additionally or alternatively, vanes may be provided in the vicinity of the inlet and outlet apertures for directing the flow of air in a desired direction. In a most preferred embodiment, there are outlet vanes that direct exiting, scented air in a direction substantially in a horizontal direction for subsequent uptake in ambient air currents.

The fan of the invention may be driven by any suitable means, although it is preferably motor-driven. The fan may take one of a number of forms including an impellers or screw-types. In a preferred embodiment however, the fan is a centrifugal fan and is arranged such that the air inlet aperture lies substantially at its centre.

Although the apparatus may be operated from mains supply electricity, a battery, solar power, a compressed gas supply or any other suitable means may also be used.

The scent vessel is preferably a container with a removable lid to prevent unwanted spillage. The lid may be of any suitable type, i.e. a screw cap or a snap-shut top.

The scent vessel may also be fitted with a perforated membrane under the screw cap. The perforated membrane may sometimes be a sponge. The size of the perforations is fine and preferably of suitable dimensions to allow sub-micron particles of the scent to escape, whilst minimising the risk of accidental spillage and contamination of body-parts or other foreign objects. The perforated membrane may also allow a larger volume of the scent to be exposed to the circulating air.

The scent vessel may comprise one or more compartments for retaining quantities of different scents. Each compartment may have a separate lid.

The scent vessel may be inserted into the chamber manually, although there is preferably provision of an inserting device. Such an inserting or loading device may be a pull out tray, a swing door or a cartridge. It is envisaged that a preferred embodiment of the invention will provide a loading tray that has formations for retaining the vessel, which can be slid or swung into the chamber. When in the chamber, the vessel or loading tray may engage with formations of the chamber to retain it in-situ until a change of scent or a replacement scent vessel is required inside the chamber, there may also be provided one or more scent vessel closing means. Such means may be a disc with a seal that clamps onto the top of the jar to prevent scent from escaping. Additionally or alternatively, a hit and miss aperture may be provided in or around such a sealing arrangement to selectively control the rate at which scent escapes from the scent vessel. Where the scent vessel comprises more than one compartment, separate closing means may be provided for each compartment.

Preferably still, the closing means is mechanised such that the rate of scent deposition may be controlled remotely. In a most preferred embodiment, the closing means is controlled by a solenoid, although this is not a limiting feature since alternative means, such as a motor or lever may equally be used. Where more than one scent vessel closing means is provided, each closing means may by independently controlled. Thus, different scents may be released at different times or a mixture of scents from more than one compartment may be released together.

The arrangement of inlet and outlet apertures, the position and direction of rotation of the fan and/or the position of the scent vessel are important in determining the efficiency of the apparatus. The scent vessel is ideally located within the chamber at a position where the airflow is fastest and/or where the air pressure is lowest. This arrangement allows the venturi effect and the evaporation of the scent to maximised respectively. In practice however, a compromise may need to be sought between the two optimum conditions.

Additionally or alternatively, stators may be provided within the chamber to maximise the aerodynamic effect of the fan and/or to direct airflow efficiently around the chamber, around the scent vessel and through the inlet and outlet apertures.

Preferably also, there is provided a circuit for controlling the operating parameters such as fan speed and direction, opening of the scent sealing means and/or control of a hit and miss aperture, where provided. The circuit may have pre-set switches for turning the fan on or off and/or for controlling the fan speed. The circuit may incorporate a timer for time-dependent operation. Additionally or alternatively, the apparatus may comprise one or more passive infra-red (PIR) sensors such that it is automatically activated or de-activated when a person approaches the apparatus. The PIR sensor or sensors may be mounted on the apparatus or may be located away from the apparatus but linked to the apparatus by way of a wire link or wireless link. Where provided, the wireless link may be a radio or infrared link. The circuit may also comprise a display panel and control buttons. Additionally or alternatively, the circuit and/or apparatus may be remotely controlled.

A preferred embodiment of the present invention shall now be described, by way of example only, with reference to the accompanying drawings, in which:

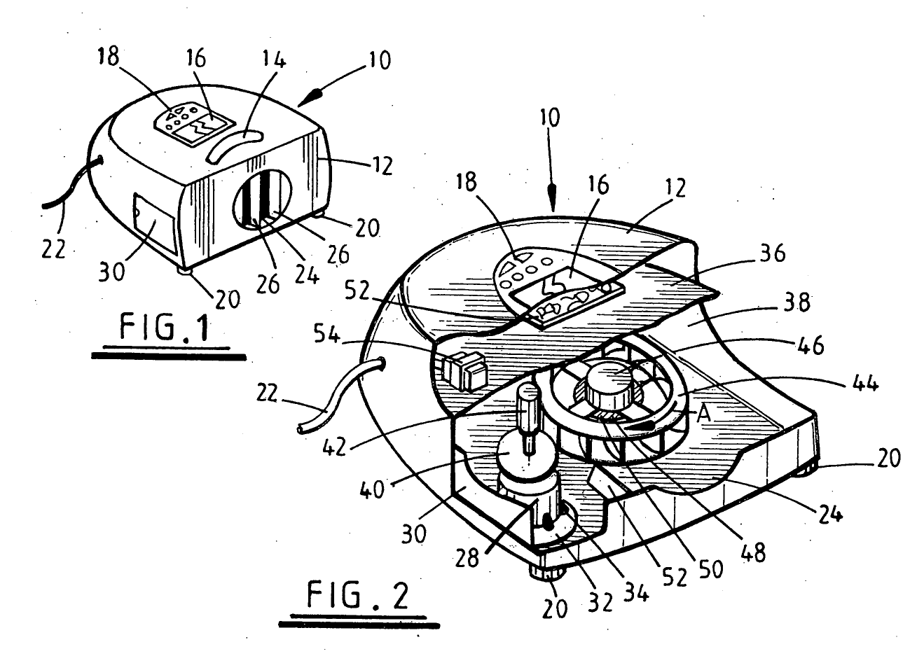

FIG. 1 is a perspective view of a preferred embodiment of the invention.

FIG. 2 shows a cut-away view of the invention revealing the arrangement of apertures, the fan and scent vessel;

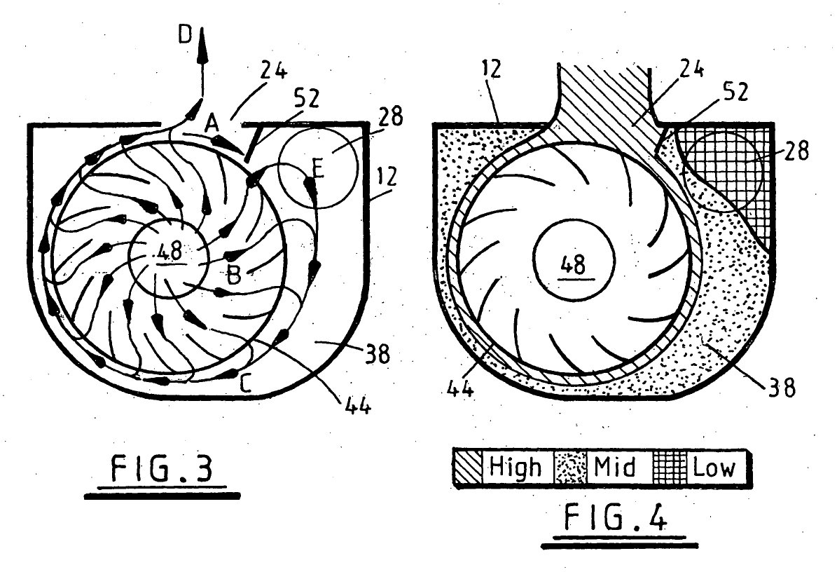

FIG. 3 illustrates schematically, the circulation of air within the chamber whilst in use;

FIG. 4 illustrates schematically, an idealised air pressure distribution map of the chamber, whilst in use;

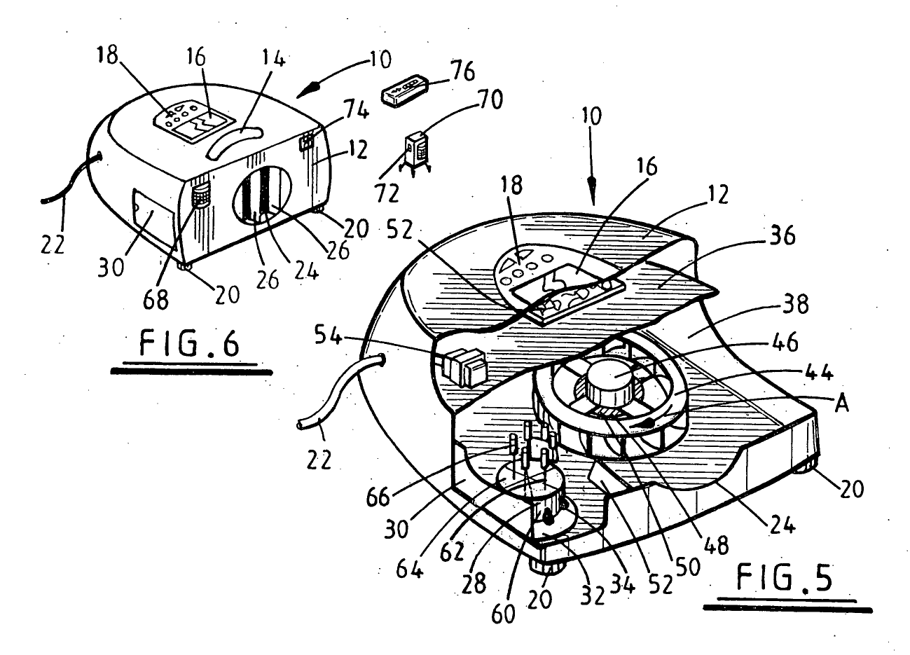

FIG. 5 shows a part cut-away perspective view of the invention having a multi-compartment scent vessel, passive infrared sensors and a remote control unit; and

FIG. 6 shows a perspective view of the invention.

Referring now to the FIGS. 1 and 2, a preferred embodiment of the invention 10 is shown. The apparatus has a body 12 with a handle 14, a display panel 16 and a control panel 18. The body 12 is variably orientated with respect to the floor by way of adjustable feet 20. The apparatus is mains powered and accordingly, has a dependent mains flex 22. The outlet aperture 24 is visible on the front of the body 12 and has vanes 26 for directing the exiting, scented airflow in a desired direction. The scent is loaded in a vessel 28 via door 30. The door 30 has a dependent tray 32 with formations 34 for retaining the vessel 28 securely. The door 30 is shown as a swing-door, although a sliding tray or other means would be equally effective.

The body 12 of the apparatus 10 has two main compartments; an upper electronics compartment 36 and a lower working chamber 38. The working chamber 38 contains the scent vessel 28 on its tray 32. The scent vessel 28, once loaded in the chamber, is sealed by a drop-down disc 40. The raising and lowering of the disc is effected by a solenoid 42. The disc 40 has an elastomeric seal on its underside for effectively sealing the vessel 28 shut. The scent vessel 28 and associated loading means 30 & 32 are located in a corner of the working chamber 38.

There is also present in the working chamber 38, a centrifugal fan 44, which is driven by a motor 46. The fan turns in the direction indicated by the arrow A.

The inlet aperture 48 is located in the centre of the centrifugal fan 44 and is protected by a grille 50.

The upper chamber 36 contains a control circuit 52 and associated electronic apparatus 54 for controlling the operation of the motor 46 and the solenoid 42.

In use, the solenoid 42 is used to raise the disc 40 thereby opening the scent vessel 28, whilst at the same time, the fan 44 is turned in the direction indicated by the arrow A. Air is thereby drawn in through the inlet aperture 48, around the chamber 38 and over the open scent vessel 28 and out through the outlet aperture 24. The operation of the solenoid 42 may be programmed to open and close the scent vessel at predetermined intervals and/or the speed of the fan 44 may also be programmed and hence, the throughput of air and scent may be varied.

Turning now to FIGS. 3 and 4, the main features i.e. the scent vessel 28, fan 44, inlet 48 and outlet apertures 24 are shown in the body 12 of the apparatus. Also shown is an optional stator 52, which although not essential for the operation of the apparatus, may improve its efficiency.

FIG. 3 shows the airflow schematically within the working chamber 38. Arrow B shows how the air is drawn through the inlet aperture 48 to the perimeter of the fan 44, whereupon it is expelled by the vanes of the fan into the chamber 38. The direction of rotation A of the fan causes the air to circulate in the direction generally referred to by the arrow C. The air is drawn around the chamber 38 and expelled D through the outlet aperture 24. A portion of the circulating air E, passes over the scent vessel 28 and picks-up scent particles by venturi action. The scent particles join the general airflow C and are expelled D through the outlet aperture 24.

Finally, FIG. 4 shows a schematic air pressure distribution map shown by the areas of hatching as labelled in the legend ‘HIGH’, ‘MID’ and ‘LOW’. The circulation of the air within the chamber causes the air pressure to be lower as you move further upstream of the outlet aperture 24. With the arrangement proposed by the present invention, the air pressure in the chamber 38 is lowest in the vicinity of the scent vessel 28, as denoted by the cross-hatching.

Further downstream, the air pressure increases as shown by the dot-hatching and is at its highest, diagonal-hatching in the vicinity of the outlet aperture 24.

In this way, the scent is helped to evaporate by the low-pressure region in which the scent vessel 28 sits and is removed from the chamber by forced air circulation and the air pressure gradient.

FIG. 5 shows largely the same features as FIG. 2, with the addition of a multiple-chamber scent vessel 60. The scent vessel 60 is shown divided into compartments 62, each compartment having a closure 64. The closures 64 are independently openable using the actuators 66.

FIG. 6 is similar to FIG. 1 but shows the apparatus 10 having passive infrared (PIR) sensor 68 located thereon. When a warm object, e.g. a person, moves in front of the apparatus 10, a message is sent to the circuit 52, which may activate or deactivate the apparatus 10, or cause the apparatus 10 to perform some other desired function. A further remote PIR sensor 70 is shown. The remote PIR sensor 70 can be positioned at a convenient location and communicates with the wireless receiver 72 of the apparatus 10 using a wireless transmitter 74. Also shown in FIG. 6 is a remote control unit 76 that communicates with the apparatus 10 via the wireless receiver 74 located on the apparatus 10.

1. An air scenting apparatus comprising; a chamber, the chamber having at least one air inlet aperture and at least one air outlet aperture; a fan arranged to draw air into the chamber through an inlet aperture and out via an air outlet aperture; and a vessel containing a scenting medium arranged to lie at least partially in the air stream between the air inlet and air outlet apertures.

2. An air scenting apparatus as claimed in claim 1, wherein the fan is a centrifugal fan.

3. An air scenting apparatus as claimed in claim 2, wherein the apparatus has a body containing a chamber, the chamber being adapted to accommodate the fan with minimal axial clearance.

4. An air scenting apparatus as claimed in claim 1, wherein the apparatus has a body, the body having at least one inlet aperture and at least one outlet aperture.

5. An air scenting apparatus as claimed in claim 4, wherein the inlet and outlet apertures have guards to prevent ingress of unwanted objects or body-parts into the chamber.

6. An air scenting apparatus as claimed in claim 1, wherein vanes are provided in the vicinity of the at least one inlet aperture for directing the flow of air in a desired direction.

7. An air scenting apparatus as claimed in claim 1, wherein vanes are provided in the vicinity of the at least one outlet aperture for directing the flow of air in a desired direction.

8. An air scenting apparatus as claimed in claim 7, wherein the vanes direct exiting, scented air in a substantially horizontal direction.

9. An air scenting apparatus as claimed in claim 1, wherein the fan is motor-driven.

10. An air scenting apparatus as claimed in claim 1, wherein the apparatus is powered by mains supply electricity.

11. An air scenting apparatus as claimed in claim 1, wherein the apparatus is powered by a battery.

12. An air scenting apparatus as claimed in claim 1, wherein the apparatus is powered by solar panel.

13. An air scenting apparatus as claimed in claim 1, wherein the scent vessel further comprises a perforated membrane.

14. An air scenting apparatus as claimed in claim 13, wherein the perforated membrane is manufactured of a foam material.

15. An air scenting apparatus as claimed in claim 1, wherein the scent vessel is divided into more than one compartment for retaining quantities of different scents.

16. An air scenting apparatus as claimed in claim 1, wherein the scent vessel has a closure.

17. An air scenting apparatus as claimed in claim 15, wherein the scent vessel has a separate closure for each compartment.

18. An air scenting apparatus as claimed in claim 1, wherein each closure comprises a hit and miss aperture to control the rate at which scent escapes from the scent vessel.

19. An air scenting apparatus as claimed in claim 16, wherein the closure or closures is/are mechanised for automated operation.

20. An air scenting apparatus as claimed in claim 19, wherein the closure or closures are mechanised using a solenoid or solenoids.

21. An air scenting apparatus as claimed in claim 20, wherein each solenoid is independently controllable.

22. An air scenting apparatus as claimed in claim 1, wherein the scent vessel is located within the chamber at a position where the airflow is fastest.

23. An air scenting apparatus as claimed in claim 1, wherein the scent vessel is located within the chamber at a position where the air pressure is lowest.

24. An air scenting apparatus as claimed in anyone claim 1, wherein one or more stators are provided within the chamber.

25. An air scenting apparatus as claimed in claim 1, further comprising a circuit for controlling operating parameters of the apparatus including:
fan speed;
fan direction;
opening of the closure or closures; and/or
control of the hit and miss aperture or apertures.

26. An air scenting apparatus as claimed in claim 25, wherein the circuit has pre-sets.

27. An air scenting apparatus as claimed in claim 25, wherein the circuit incorporates a timer for time-dependent operation.

28. An air scenting apparatus as claimed in claim 1, further comprising a passive infrared (PIR) sensor.

29. An air scenting apparatus as claimed in claim 28, wherein the PIR sensor is located away from the apparatus but linked to the apparatus by way of a wire link or wireless link.

30. An air scenting apparatus as claimed in claim 1, further comprising a display panel.

31. An air scenting apparatus as claimed in claim 1, wherein the apparatus is remotely controlled.

32. (canceled)
